(19) United States
(12) Patent Application Publication
(10) Pub. No.: US 2002/0123061 A1
(43) Pub. Date: Sep. 5, 2002

(54) Multiple use non-species specific genetic material collection and storage kit

(76) Inventors: Florrye Cleveland, Birmingham, AL (US); Leigh Harman, Hoover, AL (US)

Correspondence Address: Benjamin Aaron Adler, ADLER & ASSOCIATES, 8011 Candle Lane, Houston, TX, 77071, US

(21) Appl. No.: 10/034,292

(22) Filed: Jan. 3, 2002

Related U.S. Application Data
(63) Continuation-in-part of application No. 09/260,743, filed on Mar. 1, 1999, now abandoned.

(51) Int. Cl. C12Q 1/68; A01N 1/02

(52) U.S. Cl. 435/6; 435/2

(57) ABSTRACT

The present invention provides a method for collecting and storing non-blood genetic material from a sample in a subject by an individual comprising the steps of collecting the non-blood genetic material; transferring the non-blood genetic material to a dry solid support matrix contained on a storage card; sealing the storage card into a sample pouch; and storing the sealed sample pouch in a location designated by the individual or the subject. Also provided is a kit with which to collect non-blood genetic material.

### CROSS-REFERENCE TO RELATED APPLICATION

This continuation-in-part patent application claims benefit of priority under 35 U.S.C. 120 of non-provisional patent application U.S. Ser. No. 09/260,743, filed Mar. 1, 1999, now abandoned.

### BACKGROUND OF THE INVENTION

1. Field of the Invention

The present invention relates generally to the fields of collection and storage of genetic material. More specifically, the present invention relates to methods and materials for the instruction, collection, preservation, and storage of non-species specific genetic material.

2. Description of the Related Art

Previously, genetic material was obtained from humans in a healthcare setting by a licensed healthcare professional. A blood sample was drawn and stored in a commercial setting such as a laboratory or a hospital. This procedure greatly limited the number of people obtaining genetic material samples, compromised their privacy, and failed to address the impact of genetic testing and genetic material sample storage on non-human animal species and/or viral genetic material.

New advances in the field of genetics make the importance of having genetic material preserved clear. However, many disadvantages are present in the previous and current methods of obtaining and storing genetic material samples. For example, banking genetic material exclusively in commercial facilities is risky and has limitations. The facility could go out of business leaving the owner of the genetic material in a situation of searching for adequate storage. In addition, samples could be lost, destroyed, or switched. Therefore, with no strict guidelines in place which address many of these ethical and legal issues, storage of genetic material in commercial DNA banks has many risks.

Another problem with commercial storage is the lack of sample control by the patient. Genetic material is a source of copious amounts of confidential information. The control of the sample could be at issue at the time of death. Also at issue is the availability of the sample to insurance carriers, employers, or government agencies that could, potentially, use the sample to the detriment of the individual or future progeny. In most cases, the process is expensive, and subject to an annual fee. Allowing the patient to control collection and long-term storage would clearly be an improvement over the more expensive, ethically riddled commercial banking.

The issue of commercial storage problems was only partially solved by a home/self storage human DNA kit that allowed for the collection and storage of multiple source human DNA samples for future genetic testing. Bierke-Nelson et al. (U.S. Pat. No. 5,856,102) discloses a method for home/self storage of human DNA. However, the method is limited to human DNA collection and offers no instruction for the collection of genetic material from other animal species.

The Bierke-Nelson kit is based on the recommendation of the collection of multiple source human DNA samples. However, the underlying premise of multiple source necessity is questionable. Most genetic diseases are inherited through germline mutations. Somatic mutations are generally not inherited in subsequent generations, nor are they applicable for identification. It is also recommended that a blood sample be obtained and stored on a card at home. This requires collection by a healthcare professional thus, not only increasing cost and inconvenience, but also compromising privacy.

The Bierke-Nelson kit does not overcome certain storage problems. Human DNA is put on brushes/swabs and stored in the home at room temperature. No precaution is provided to decrease sample degradation or prevent other environmental insults. Samples stored untreated at room temperature under humid conditions are at risk for contamination and degradation. No provision is made for the application of the genetic material to a dry solid medium that is subsequently stored. Furthermore, the Bierke-Nelson kit does not use tamper-resistant packaging that could lead to compromise privacy and/or integrity of the sample.

Patents (U.S. Pat. Nos. 5,101,970 (1992) and 5,211,286 (1993)) were issued to Turner for a home kit for the collection and preservation of information for identifying and locating lost or missing relatives. Turner's kit requires collection of multiple samples, such as human hair, blood, and fingerprints, and is, therefore, limited to human samples with no provision for sampling of non-human or viral genetic material.

With the Turner kit, all samples are stored in a freezer, leaving no option for room temperature storage. Furthermore, genetic material samples from up to four different people are placed in a closed container for long-term storage. This could result in cross-contamination of samples thus potentially leading to ineffective genetic analysis.

Current technology allows genetic typing for a variety of reasons, e.g., identification, disease diagnosis or prediction, forensic testing, and viral identification. A kit that provides a confidential, convenient, and reliable method for collecting and storing genetic material would, therefore, be beneficial. The inventors have recognized a need for a simple, easy to use and convenient collection and room-temperature storage method of non-blood genetic material

The prior art is deficient in the lack of an easy-to-use multi-use genetic material collection and storage kit. Specifically, there is need of a genetic material collection and storage kit that facilitates the collection and long-term storage of genetic material from humans, animals and/or viruses. The present invention fulfills this long-standing need and desire in the art.

### SUMMARY OF THE INVENTION

In one embodiment of the present invention there is provided a method for collecting and storing a non-blood genetic material sample, said sample collected from a subject by an individual, comprising the steps of collecting the sample; transferring the sample to a dry solid support matrix contained on a storage card; sealing the storage card into a sample pouch; and storing the sealed sample pouch in a location designated by the individual or the subject.

In another embodiment of the present invention there is provided a method of collecting and storing a non-blood genetic material sample, said sample collected from a human or non-human animal subject by an individual wherein the individual is optionally the subject, comprising the steps of labeling a storage card wherein the storage card comprises one or more specimen circles containing the dry solid support matrix; swabbing the inside of the mouth of the subject with a sponge to obtain a non-blood genetic material sample comprising mouth cells, saliva, viruses, infectious organisms or a combination thereof wherein the sample sorbs onto the sponge; blotting the sponge containing the sample sorbed thereon onto a specimen circle comprising the labeled dry solid support matrix on the storage card wherein the sample is affixed to the dry solid support matrix; air-drying the affixed sample onto the labeled dry solid support matrix wherein the dry solid support matrix comprises a compound or composition that reduces the risk of contamination and degradation of the affixed non-blood genetic material; labeling a resealable storage pouch; placing the storage card containing the sample affixed to the dry solid matrix into the resealable storage pouch wherein the storage pouch contains a desiccant; closing the resealable storage pouch; placing a tamper-resistant security tape over the closed portion of the resealable storage pouch thereby sealing it; labeling a permanent storage record folder; placing the sealed storage pouch into the labeled permanent storage record folder; and storing the labeled permanent storage record folder in a location designated by the individual or the subject wherein the sample affixed to the dry solid support matrix is obtainable at any time by the individual or the subject for subsequent analysis.

In yet another embodiment of the present invention there is provided a kit for collecting non-blood genetic material, comprising a sponge; a dry solid support matrix; a dessicant; a storage pouch; and a permanent storage record folder.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 is a detailed listing of the instructions to use the non-blood genetic material collection and storage kit.

FIG. 2 depicts the identification and beneficiary forms contained in the non-blood genetic material collection and storage kit.

### DETAILED DESCRIPTION OF THE INVENTION

In one embodiment of the present invention there is provided a method for collecting and storing a non-blood genetic material sample, said sample collected from a subject by an individual, comprising the steps of collecting the sample; transferring the sample to a dry solid support matrix contained on a storage card; sealing the storage card into a sample pouch; and storing the sealed sample pouch in a location designated by the individual or the subject.

The individual collecting the sample may be the subject or the subject may be a non-human subject such as animals. The non-blood genetic material collected may be saliva, mouth cells, or a combination thereof. Additionally, the genetic material may be from viruses and infectious organisms contained in saliva and/or mouth cells. The sample is protected from degradation and contamination by using a dry solid matrix having a compound or composition that reduces this risk.

In this embodiment collecting the non-blood genetic material from the subject comprises the steps of labeling the storage card wherein the storage card comprises one or more specimen circles containing the dry solid support matrix; and swabbing the inside of the mouth of the subject with an absorbent material wherein the sample sorbs onto the absorbent material. A representative example of absorbent material is a sponge.

In this embodiment transferring the non-blood genetic material sample comprises the steps of blotting the absorbent material containing the sample sorbed thereon onto the specimen circle comprising the labeled dry solid support matrix on the storage card where the sample is affixed to the dry solid support matrix; and air-drying the affixed sample onto the dry solid support matrix. Additionally, blotting and transferring of the genetic material is repeated until all of the at least one specimen circles have the genetic material affixed thereto.

In this embodiment sealing the storage pouch comprises the steps of labeling the storage pouch; placing the storage card containing the sample affixed to the dry solid matrix into the storage pouch where the storage pouch optionally contains a desiccant; closing the storage pouch; and placing a tape over the closed portion of the storage pouch. In one aspect, the storage pouch is resealable and the tape is a tamper-resistant security tape.

In this embodiment storing the sealed storage pouch comprises the steps of labeling a permanent storage folder; placing the sealed sample pouch into the permanent storage folder; and storing the labeled permanent storage record folder in a location designated by the individual or the subject wherein the sample affixed to the dry solid support matrix is obtainable at any time by the individual or the subject for subsequent analysis.

In another embodiment of the present invention there is provided a method of collecting and storing a non-blood genetic material sample, said sample collected from a human or non-human animal subject by an individual wherein the individual is optionally the subject, comprising the steps of labeling a storage card wherein the storage card comprises one or more specimen circles containing the dry solid support matrix; swabbing the inside of the mouth of the subject with a sponge to obtain a non-blood genetic material sample comprising mouth cells, saliva, viruses, infectious organisms or a combination thereof wherein the sample sorbs onto the sponge; blotting the sponge containing the sample sorbed thereon onto a specimen circle comprising the labeled dry solid support matrix on the storage card wherein the sample is affixed to the dry solid support matrix; air-drying the affixed sample onto the labeled dry solid support matrix wherein the dry solid support matrix comprises a compound or composition that reduces the risk of contamination and degradation of the affixed non-blood genetic material; labeling a resealable storage pouch; placing the storage card containing the sample affixed to the dry solid matrix into the resealable storage pouch wherein the storage pouch contains a desiccant; closing the resealable storage pouch; placing a tamper-resistant security tape over the closed portion of the resealable storage pouch thereby sealing it; labeling a permanent storage record folder; placing the sealed storage pouch into the labeled permanent storage record folder; and storing the labeled permanent storage record folder in a location designated by the individual or the subject wherein the sample affixed to the dry solid support matrix is obtainable at any time by the individual or the subject for subsequent analysis. Additionally, blotting and transferring of the genetic material is repeated until all of the at least one specimen circles have the genetic material affixed thereto.

In yet another embodiment of the present invention there is provided a kit for collecting non-blood genetic material, comprising a sponge; a dry solid support matrix; a dessicant; a storage pouch; and a permanent storage record folder. The kit may additionally contain tape. A representative example of this tape is a tamper-resistant security tape.

Provided herein is an improved process and method of genetic material collection and storage in which genetic material is privately collected and stored in a setting chosen by the collector. The invention includes the kit that is designed to collect a single source genetic material sample from many animal species and/or viruses, and a method to bank/keep the genetic material sample in a setting chosen by the collector.

The instant kit is simple to use. A genetic material sample comprising saliva, mouth cells or a combination thereof is absorbed from the mouth of the subject onto a sponge, or other absorbent device, by the collector who swabs the mouth of the subject using the sponge or other device. This procedure can be easily self-administered, or if desired, administered by another person. The genetic material sample is then affixed to a dry solid matrix.

The sample is then dried on the dry solid matrix, sealed, labeled and stored in a location chosen by the collector or mailed for analysis in an enclosed, water-resistant, puncture-resistant permanent storage folder. The choice of storage is at the discretion of the collector. Since no blood is needed, no healthcare provider is required, thus insuring confidentiality and convenience. The kit provides detailed instructions for an untrained individual to collect and store samples from humans and many animal species. In fact, one does not have to have any particular skill in the art to practice the instant invention.

The improvements over prior art kits include, but are not limited to:

Confidentiality

This method of collection has the option of complete confidentiality. Saliva and mouth cells are collected with a sponge swab and placed on a dry solid matrix. Other genetic sample kits use a process whereby blood is drawn and placed on a card for storage, or a brush is used to store cheek cells for later analysis. Due to the dry solid matrix in the kit, a genetic sample is obtained from the saliva and mouth cells and stored on the matrix in a private location, if desired. Thus, since no blood sample is drawn, collection of the sample by a licensed healthcare professional is not required and, therefore, confidentiality is assured.

Privacy

The kit provides a means for insuring privacy. Privacy is protected since the collector controls sample collection. This in-home collection process allows the collector to confidentially obtain a genetic material sample for a number of reasons, including, but not limited to, screening for infectious diseases. A genetic sample is collected in a private setting and confidentially sent for testing or stored for future analysis.

Sample Preservation

In the instant kit the matrix and not the sponge or other collection device is stored. The non-blood genetic material sample is self-collected and is stored on a solid matrix having a compound or composition that protects against degradation and/or contamination of the genetic material sample. Also, no special requirements for storage, such as refrigeration or freezing are required.

Control of the Sample

Personal ownership of the sample provides control over who has access to the sample. The sample is not stored at a commercial facility thus avoiding potential problems due to, for example, unauthorized access, inadequate record keeping, inadvertent loss of the sample and storage fees. Therefore, since the sample is stored at a location chosen by the collectors there exists less chance of a sample mix-up or loss.

Storage Options

Placement of the genetic sample is at the collector's discretion although it is contemplated that such storage is in-home and/or self-storage. The ease of acquiring the sample as described herein allows the collector to obtain a sample at a time and location of his choice. The collector has the option of long-term storage of the sample, or immediately mailing the sample for analysis.

The kit provides storage under a tamper-resistant seal on the storage pouch. This is important to document any handling of the sample and insures security of the sample. Additionally, a water-resistant, puncture-resistant storage folder is provided within which to place the sealed storage pouch. This storage folder can be mailed immediately or if and when the collector chooses to analyze the sample. The sample can be stored at room temperature in any location desired.

The kit is designed for the collection and the long- or short-term private storage on a dry solid medium of a non-blood genetic material sample. The kit can be used for collection and private storage of genetic material from large and/or small domesticated and non-domesticated animals for parentage validation, pedigree analysis, and disease identification. The kit provides for the simple collection of a genetic material sample from many animal species and/or viruses, either in their natural habitat or in a convenient location. Storage of the non-blood genetic material sample by the methods disclosed herein has the lowest risk of bacterial contamination and environmental insult for a non-blood genetic material sample. Although not limited to those disclosed, some of the preferred objects of the kit are:

Determining Parentage or Ownership in Animals

Many species of animals can be tested to determine their parentage. For example, a prospective purchaser could obtain a genetic material sample from an animal and its parents. The samples could be tested to determine if that animal is, in fact, an offspring of those parents. It is contemplated that breeders and/or purchasers or of domestic and non-domestic animals would use the instant kit. Additionally, animal owners would have a way to positively identify their animals in cases of loss or theft.

Disease Diagnosis or Prediction

Disease diagnosis or prediction in animals would be possible. The ease of obtaining the genetic material sample from saliva means that a high-quality animal sample could be collected anywhere, even in the animal's natural habitat, to determine if it is a carrier of any genetic mutation or viral infection.

Genetic History of Adopted Children

Genetic material samples could be collected from adopted children and their biological families. Since the kit only requires a single source of genetic material, a sample could easily, quickly, and inexpensively be obtained on any willing biological family members of the child to be adopted. Attorneys and/or adoption agencies could store the genetic sample from the biological family members for such things as future use in genetic testing, for determination of heredity or identification of siblings or other birth family relatives should the birth family allow it. To protect the privacy of the biological family, the samples could simply be listed as biological mother, biological father, biological maternal grandmother, biological paternal grandfather, etc. Thus, the biological family's privacy would bot be compromised, and yet the adopted child would have invaluable genetic information.

Determination of Identity for Legal Issues

The kit provides a means for positive identification in legal matters. Attorneys could have the ability to store positive identification on clients in estate settlement cases. Because the kit uses a simple method of collection and storage of a genetic material sample, the attorney could store the client's sample for future identification. Therefore, anyone claiming to be a relative of the client could be included or excluded based on his/her genetic link with the estate.

Additionally, an attorney is able to confidentially collect a sample from a client to compare with other genetic material the opposing counsel could have on that same client. This would give the client's attorney her own proof as to the validity or not of other potential genetic evidence.

Identification of Children

Parents can have positive identification on their children. Parents can easily collect and store genetic material samples on their offspring as a means of future identification. This could be critical in identifying decomposed remains and helping officials solve missing persons' cases.

Genetic Prediction and Risk Assessment

Families can have genetic information on previous generations for genetic diagnosis and prediction of risk assessment. Genetic material samples taken on current generations maybe critical for piecing together the genetic puzzle for future generations.

Analysis by Physicians, Dentists and Veterinarians

With the subject's consent physicians, dentists, and veterinarians can collect samples for genetic analysis and storage. The samples can be used for screening for diseases, as diagnostic tools, for identification or may just be stored in the office as part of the subject's record.

Genealogical Research

The kit provides a means for easing the process for people who begin a search for their past through genealogical records. In a potential application of the kit it is contemplated that individuals could match themselves with distant relatives. In many instances records are hard to piece together, however, many questions can be answered by comparing genetic material. This kit could help these individuals collect genetic material for potential genetic testing to prove heritage.

The following examples are given for the purpose of illustrating various embodiments of the invention and are not meant to limit the present invention in any fashion.

### EXAMPLE 1

Components of the Collection Kit

The components of a kit designed to employ these procedures include, but are not limited to:

A Sponge

Collection of the sample(s) is effected by a sponge and/or any other device designed to collect saliva and mouth cells. For example, provided in the instant kit is a sterile foam-tipped sponge with a handle protectively sealed in paper to maintain sterility. The applicator is about 6 inches in total length with a handle of about 4 inches in length. An example of such a sponge is provided by Pur-Wraps®. Additionally, a latex glove is provided that can be worn during collection of the sample.

A Dry Solid Matrix and a DNA Storage Card

A card having four specimen circles each comprising the dry solid matrix is provided. The dry solid matrix comprises a compound or a composition that resists contamination and degradation of the non-blood genetic material affixed thereon. Until use the storage card is kept sealed in a water-resistant, puncture-resistant bag; the bag must be cut to be opened thereby maintaining the integrity of the dry solid matrix.

The card is about 6.5 inches in length by about 2 inches in width and folds lengthwise in about thirds similar to a book of matches thus enclosing and protecting the dry solid matrix prior to and after sample collection and transfer. The front of the folded storage card can be labeled. The back of the folded storage card has instructions as provided on the permanent storage folder printed thereon.

Sample Storage Pouch

A storage pouch having a segment for labeling is provided for storing the dry solid matrix containing the non-blood genetic material. The storage pouch is about 7.0 inches long by about 4.0 inches wide. The labeling area on the front of the storage pouch is about 2.5 inches long by about 4.0 inches wide. Any type of marker or pen, optimally containing waterproof and smudge-resistant ink, can be used to label the sample storage pouch. This insures tamper-resistant labeling.

The storage pouch is closed at the top with a “ziplock-type” resealable means. Additionally, there is provided a tape to seal the storage pouch. Such a tape is a tamper-resistant security tape such as can be provided by, for example, Lab Safety Supply Inc. After the DNA storage card is placed in the storage pouch and the pouch closed, the security tape is placed over the closed storage pouch in such a way that opening the pouch by normal means would damage or tear the tape or damage or tear the storage pouch or both. Optionally, the storage pouch can contain a dessicant. Although not limited to said, an example of such a dessicant is SORB-IT®.

Permanent Storage Folder

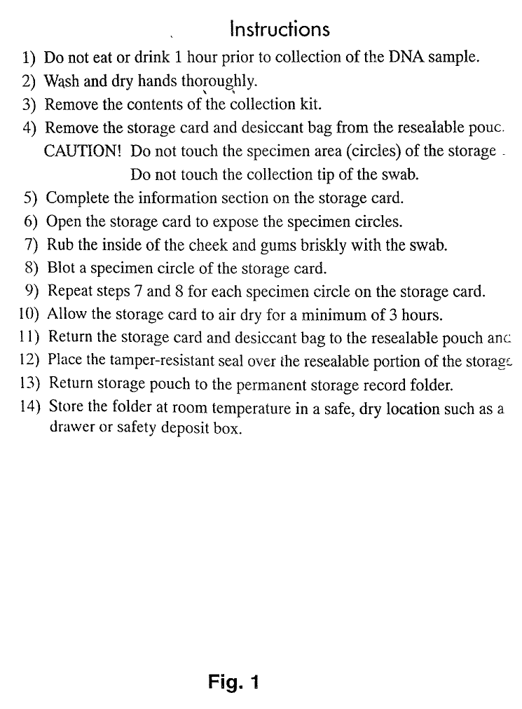

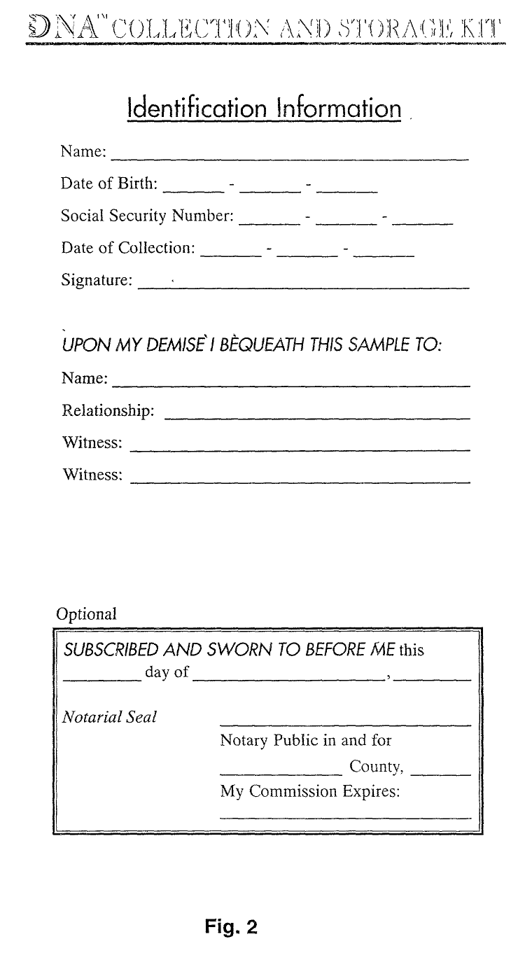

The kit contains a water-resistant, puncture-resistant permanent storage folder about 12.0 inches wide×about 8.5 inches long when opened; the folder is folded in half width wise. The inside right-hand side of the folder contains a pocket that contains the supplies disclosed supra before use of the kit. Additionally, to insure the kit is used properly, instructions are printed on the front of the pocket (FIG. 1) contained on the inside right-hand side of the storage folder. The inside left-hand side of the storage folder contains areas to provide sample identification information and to designate a beneficiary (FIG. 2).

After collection of the sample, it is intended that the sealed sample storage pouch containing the DNA storage card be placed inside the pocket in the permanent storage folder. The folder is closed and may be either stored at room temperature in any location designated by the owner of the sample or may be immediately mailed for analysis. The storage folder may be mailed by placing it in any sturdy conventional envelope of a size sufficient to easily accommodate the permanent storage folder.

### EXAMPLE 2

Collection Process

Specific instructions to use the collection kit for collecting a non-blood genetic material sample and for handling of the sample after collection are listed in FIG. 1. Prior to collection of the sample, the contents of the permanent storage folder are removed and the DNA storage card, the sample storage pouch and the permanent storage folder are labeled. The DNA storage card is then opened to expose the specimen circles without touching the specimen circles. A sponge, as disclosed, is removed from its paper wrapper by the handle and is used as a swab to collect saliva and mouth cells from the mouth of the subject by rubbing the inside of the cheek and gums of the subject briskly with the swab/sponge.

Following collection of the sample, and using specific instructions enclosed in the kit, the sponge is blotted to a specimen circle containing the dry solid matrix on the labeled DNA storage card. This procedure of swabbing to collect the sample and transferring the sample to a specimen circle is repeated until all four of the specimen circles have a sample transferred thereon. The samples transferred to the dry solid matrix are air-dried for at least about 3 hours.

Again following provided instructions, the DNA storage card containing the four specimens is folded and placed in the labeled sample storage pouch with the dessicant. The sample storage pouch is closed and sealed with a tamper-resistant security tape that is placed over the resealable portion of the storage pouch. The sample storage pouch is then placed in the pocket of the water-resistant, puncture-resistant permanent storage folder. The permanent storage folder is suitable for indefinite storage in a secure place or suitable for mailing the sample for analysis.

The description of the kit and methods used to collect the non-blood genetic material described supra should not be construed as limiting the scope of the invention but as merely providing illustrations of some of the preferred embodiments of this invention. It is contemplated that the collection device can be altered, i.e., the size, shape, and design of the device designed to collect the saliva and mouth cells could vary or even possibly be eliminated. Furthermore, it is contemplated that the subject need only lick the matrix or a similar substance to obtain a sample.

The storage of the non-blood genetic material is not restricted to the means disclosed herein. The size and shape of the storage pouch could vary or be eliminated or a different type of pouch could be used. The tamper-resistant labels could be in a different form or eliminated. Instructions and forms could vary. Therefore, the kit, the items in the kit and the method of using the kit can be altered as needed to accommodate the best mode and the latest technology.

Any patents or publications mentioned in this specification are indicative of the levels of those skilled in the art to which the invention pertains. These patents and publications are herein incorporated by reference to the same extent as if each individual publication was indicated to be incorporated specifically and individually by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A method for collecting and storing a non-blood genetic material sample, said sample collected from a subject by an individual, comprising the steps of:
(a) collecting the sample;
(b) transferring the sample to a dry solid support matrix contained on a storage card;
(c) sealing the storage card into a sample pouch; and
(d) storing the sealed sample pouch in a location designated by the individual or the subject.

2. The method of claim 1, wherein the individual is the subject.

3. The method of claim 1, wherein the sample comprises saliva, mouth cells, or a combination thereof.

4. The method of claim 3, wherein the sample comprises genetic material selected from the group consisting of viruses and infectious organisms.

5. The method of claim 1, wherein the subject is selected from the group consisting of human and non-human animal species.

6. The method of claim 1, wherein the dry solid support matrix comprises a compound or composition that reduces the risk of contamination and degradation of the non-blood genetic material.

7. The method of claim 1, wherein collecting the non-blood genetic material sample from the subject comprises the steps of:
(a) labeling the storage card wherein the storage card comprises one or more specimen circles containing the dry solid support matrix; and
(b) swabbing the inside of the mouth of the subject with an absorbent material wherein the sample absorbs onto the absorbent material.

8. The method of claim 7, wherein the absorbent material is a sponge.

9. The method of claim 1, wherein transferring the non-blood genetic material sample comprises the steps of:
(a) blotting the absorbent material containing the sample sorbed thereon onto the specimen circle comprising the labeled dry solid support matrix on the storage card wherein the sample is affixed to the dry solid support matrix; and
(b) air-drying the affixed sample onto the dry solid support matrix.

10. The method of claim 1, wherein sealing the storage pouch comprises the steps of:
(a) labeling the storage pouch;
(b) placing the storage card containing the sample affixed to the dry solid matrix into the storage pouch wherein the storage pouch optionally contains a desiccant;
(c) closing the storage pouch; and
(d) placing a tape over the closed portion of the storage pouch.

11. The method of claim 10, wherein the storage pouch is resealable.

12. The method of claim 10, wherein the tape is a tamper-resistant security tape.

13. The method of claim 1, wherein storing the sealed storage pouch comprises the steps of:
(a) labeling a permanent storage folder;
(b) placing the sealed sample pouch into the permanent storage folder; and
(c) storing the labeled permanent storage record folder in a location designated by the individual or the subject wherein the sample affixed to the dry solid support matrix is obtainable at any time by the individual or the subject for subsequent analysis.

14. The method of claim 1, further comprising the step of:
repeating steps (a) and (b) until all of the at least one specimen circles comprising the dry solid support matrix contain the sample affixed thereto.

15. A method of collecting and storing a non-blood genetic material sample, said sample collected from a human or non-human animal subject by an individual wherein the individual is optionally the subject comprising the steps of:
(a) labeling a storage card wherein the storage card comprises one or more specimen circles containing the dry solid support matrix;
(b) swabbing the inside of the mouth of the subject with a sponge to obtain a non-blood genetic material sample comprising mouth cells, saliva, viruses, infectious organisms or a combination thereof wherein the sample sorbs onto the sponge;
(c) blotting the sponge containing the sample sorbed thereon onto a specimen circle comprising the labeled dry solid support matrix on the storage card wherein the sample is affixed to the dry solid support matrix;
(d) air-drying the affixed sample onto the labeled dry solid support matrix wherein the dry solid support matrix comprises a compound or composition that reduces the risk of contamination and degradation of the affixed non-blood genetic material;
(e) labeling a resealable storage pouch;
(f) placing the storage card containing the sample affixed to the dry solid matrix into the resealable storage pouch wherein the storage pouch contains a desiccant;
(g) closing the resealable storage pouch;
(h) placing a tamper-resistant security tape over the closed portion of the resealable storage pouch thereby sealing it;
(i) labeling a permanent storage record folder;
(j) placing the sealed storage pouch into the labeled permanent storage record folder; and
(k) storing the labeled permanent storage record folder in a location designated by the individual or the subject wherein the sample affixed to the dry solid support matrix is obtainable at any time by the individual or the subject for subsequent analysis.

16. The method of claim 15, further comprising the step of:
repeating steps (a) and (b) until all of the at least one specimen circles comprising the dry solid support matrix contain the non-blood genetic material affixed thereto.

17. A kit for collecting non-blood genetic material, comprising:
(a) a sponge;
(b) a dry solid support matrix;
(c) a dessicant;
(d) a storage pouch; and
(e) a permanent storage record folder.

18. The kit of claim 17, wherein the storage pouch is resealable.

19. The kit of claim 17, further comprising:
(f) tape.

20. The kit of claim 19, wherein the tape is tamper-resistant security tape.
